(19) United States
(12) Patent Application Publication
(10) Pub. No.: US 2009/0163494 A1
(43) Pub. Date: Jun. 25, 2009

(54) PROTEIN KINASE INHIBITORS

(76) Inventors: Pao-Chiung Hong, Xizhi City (TW); Li-Jung Chen, Xizhi City (TW); Yann-Yu Lu, Xizhi City (TW); Tzu-Yun Lai, Xizhi City (TW); Huei-Ru Yang, Xizhi City (TW); Yi-Feng Kao, Xizhi City (TW); Kuei-Tai Lai, Xizhi City (TW); Young-Sun Lin, Xizhi City (TW)

Correspondence Address: OCCHIUTI ROHLICEK & TSAO, LLP, 10 FAWCETT STREET, CAMBRIDGE, MA, 02138, US

(21) Appl. No.: 11/960,059

(22) Filed: Dec. 19, 2007

(51) Int. Cl. A61K 31/5377; C07D 413/02; C07D 403/02; C07D 209/32; A61P 35/00; A61K 31/4155; A61K 31/404

(52) U.S. Cl. 514/235.2; 514/397; 514/418; 514/414; 544/144; 548/312.1; 548/465; 548/484

(57) ABSTRACT

This invention relates to selenophene compounds of formula (I) shown below:

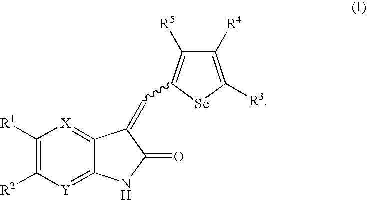

Each variable in formula (I) is defined in the specification. These compounds can be used to treat cancer.

### BACKGROUND

Many protein kinases, e.g., check-point kinase 1 (Chk1), play important roles in controlling cell cycle progression, thereby regulating cell proliferation. Overly active or inactive, they fail to control cell cycle, resulting in hyperproliferative diseases, such as cancer. Blocking or enhancing their activity can restore the control of cell cycle progression and consequently cell proliferation. Thus, protein kinases involved in cell-cycle regulation are potential therapeutic targets for treating hyperproliferative diseases.

### SUMMARY

This invention is based on the discovery that certain selenophene compounds inhibit Chk1 kinase, and thus are effective in treating cancer.

In one aspect, this invention relates to selenophene compounds of formula (I):

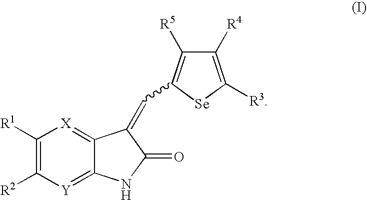

In this formula, each of X and Y, independently, is N or CR′, in which R′ is H, halo, alkyl, cycloalkyl, aryl, heteroaryl, heterocycloalkyl, OH, alkoxyl, aryloxyl, —C(O)R^{a}, —NR^{b}R^{c}, —C(O)NR^{b}R^{c}, —(CH₂)ₙC(O)R^{a}, —(CH₂)ₙNR^{b}R^{c}, or —(CH₂)ₙC(O)NR^{b}R^{c}, in which R^{a} is H, OH, alkoxyl, or aryloxyl, each of R^{b} and R^{c}, independently, is H, alkyl, cycloalkyl, aryl, or heteroaryl, or R^{b} and R^{c} together with the N atom to which they are attached are heterocycloalkyl, and n is 1, 2, 3, or 4; each of R¹ and R², independently, is H, halo, alkyl, OH, alkoxyl, aryl, heteroaryl, CN, —NR^{a1}R^{b1}, —NR^{a1}C(O)R^{b1}, —C(O)R^{c1}, —NR^{a1}S(O)₂R^{b1}, —S(O)₂NR^{a1}R^{b1}, or —SO₂R^{d1}, in which each of R^{a1} and R^{b1}, independently, is H, alkyl, cycloalkyl, heterocycloalkyl, aryl, or heteroaryl, or R^{a1} and R^{b1} together with the N atom or the N and C atoms or the N and S atoms to which they are attached are heterocycloalkyl, R^{c1} is H, OH, alkoxyl, or aryloxyl, and R^{d1} is alkyl, aryl, or heteroaryl; and each of R³, R⁴, and R⁵, independently, is H, halo, alkyl, OH, alkoxyl, aryloxyl, —C(O)R^{a2}, or —C(O)NR^{b2}R^{c2}, in which R^{a2} is OH, alkoxyl, aryloxyl, or heterocycloalkyl, and each of R^{b2} and R^{c2}, independently, is H, alkyl, cycloalkyl, aryl or heteroaryl, or R^{b2} and R^{c2} together with the N atom to which they are attached are heterocycloalkyl, or R³ and R⁴ together with the C atoms to which they are attached are cycloalkyl or heterocycloalkyl, or R⁴ and R⁵ together with the C atoms to which they are attached are cycloalkyl or heterocycloalkyl.

One subset of the above-described selenophene compounds includes those in which both of X and Y are CH. Preferably, R¹ is H and R² is phenyl optionally substituted with OH, alkoxyl, aryloxyl, or alkyl; or one of R¹ and R² is halo (e.g., fluoro) and the other is H.

Another subset of the selenophene compounds includes those in which at least one of X and Y is N. Preferably, R¹ is H and R² is phenyl optionally substituted with OH, alkoxyl, aryloxyl, or alkyl; or one of R¹ and R² is halo (e.g., fluoro) and the other is H.

Still another subset of the selenophene compounds includes Z-isomers thereof. In these compounds, both of X and Y can be CH; R¹ can be H and R can be phenyl optionally substituted with OH, alkoxyl, aryloxyl, or alkyl; one of R¹ and R² can be halo (e.g., fluoro) and the other can be H; R² can be 4-hydroxyphenyl optionally substituted with OCH₃ or 4-methoxyphenyl optionally substituted with OCH₃; each of R³, R⁴, and R⁵, independently, can be H, CH₃, alkyl optionally substituted with OH or OCH₃, —COOH, —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}, in which m can be 2 or 3, R^{a3} can be H, OH, alkyl, alkoxyl, aryloxyl, or heterocycloalkyl, and each of R^{b3} and R^{c3} , independently, can be H, alkyl, cycloalkyl, aryl, or heteroaryl, or R^{b2} and R^{c2} together with the N atom to which they are attached can be heterocycloalkyl or heteroaryl; at least one of R³, R⁴, and R⁵ can be H and at least one of the others can be —COOH or —C(O)NH(CH₂)ₘNR^{b3}R^{c3}; or at least one of R³, R⁴, and R⁵ can be H and at least one of the other of R³, R⁴, and R⁵ can be —C(O)NR^{b2}R^{c2} in which R^{b2} and R^{c2} together with the N atom to which they are attached can be heterocycloalkyl.

The term “alkyl” herein refers to a straight or branched hydrocarbon containing 1-20 carbon atoms (e.g., C₁-C₁₀). Examples of alkyl groups include, but are not limited to, methyl, ethyl, n-propyl, i-propyl, n-butyl, i-butyl, and t-butyl. The term “alkoxyl” refers to an —O-alkyl. The term “cycloalkyl” refers to a non-aromatic cyclic hydrocarbon moiety containing 3-30 carbon atoms (e.g., C₃-C₂₀), such as cyclohexyl or cyclohexen-3-yl. The term “heterocycloalkyl” refers to a non-aromatic cyclic moiety having at least one ring heteroatom (e.g., N, O, or S) containing 1-30 carbon atoms (e.g., C₁-C₂₀), such as 4-tetrahydropyranyl. The term “aryl” refers to a hydrocarbon moiety having one or more aromatic rings. Examples of aryl moieties include phenyl, phenylene, naphthyl, naphthylene, pyrenyl, anthryl, and phenanthryl. The term “aryloxyl” refers to an —O-aryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl. The term “halo” or “halogen” includes fluoro, chloro, bromo, and iodo.

Alkyl, alkoxyl, cycloalkyl, heterocycloalkyl, aryl, aryloxyl and heteroaryl mentioned herein include both substituted and unsubstituted moieties, unless specified otherwise. Possible substituents on cycloalkyl, heterocycloalkyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₁-C₂₀ heterocycloalkyl, C₁-C₁₀ alkoxyl, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on alkyl include all of the above-recited substituents except C₁-C₁₀ alkyl. Cycloalkyl, heterocycloalkyl, aryl, and heteroaryl can also be fused with each other.

In another aspect, this invention features a method for treating cancer, e.g., lung cancer, melanoma, hepatoma, leukemia, gastrointestinal stromal tumors, breast cancers, prostate cancers, renal cell carcinoma. The method includes administering to a subject in need thereof an effective amount of one or more selenophene compounds of formula (I) shown above. The term “treating” or “treatment” refers to administering one or more selenophene compounds to a subject, who has cancer, a symptom of such cancer, or a predisposition toward such cancer, with the purpose to confer a therapeutic effect, e.g., to cure, relieve, alter, affect, ameliorate, or prevent the above-described cancer, the symptom of it, or the predisposition toward it. “An effective amount” refers to the amount of one or more active selenophene compounds which is required to confer a therapeutic effect on a treated subject.

In addition, this invention encompasses a pharmaceutical composition that contains an effective amount of at least one of the above-mentioned selenophene compounds and a pharmaceutically acceptable carrier.

The selenophene compounds described above include the compounds themselves, as well as their salts, prodrugs, and solvates, if applicable. A salt, for example, can be formed between an anion and a positively charged group (e.g., amino) on a selenophene compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a selenophene compound. Suitable cations include sodium ion, potassium ion, magnesium ion, calcium ion, and an ammonium cation such as tetramethylammonium ion. The selenophene compounds also include those salts containing quaternary nitrogen atoms. Examples of prodrugs include esters and other pharmaceutically acceptable derivatives, which, upon administration to a subject, are capable of providing active selenophene compounds. A solvate refers to a complex formed between an active selenophene compound and a pharmaceutically acceptable solvent. Examples of pharmaceutically acceptable solvents include water, ethanol, isopropanol, ethyl acetate, acetic acid, and ethanolamine.

Also within the scope of this invention is a pharmaceutical composition containing one or more of the above-described selenophene compounds for use in treating cancer, as well as this therapeutic use and use of the compounds for the manufacture of a medicament for treating cancer.

The details of one or more embodiments of the invention are set forth in the description below. Other features, objects, and advantages of the invention will be apparent from the description and from the claims.

### DETAILED DESCRIPTION

Shown below are exemplary compounds, Compounds 1-57, of this invention.

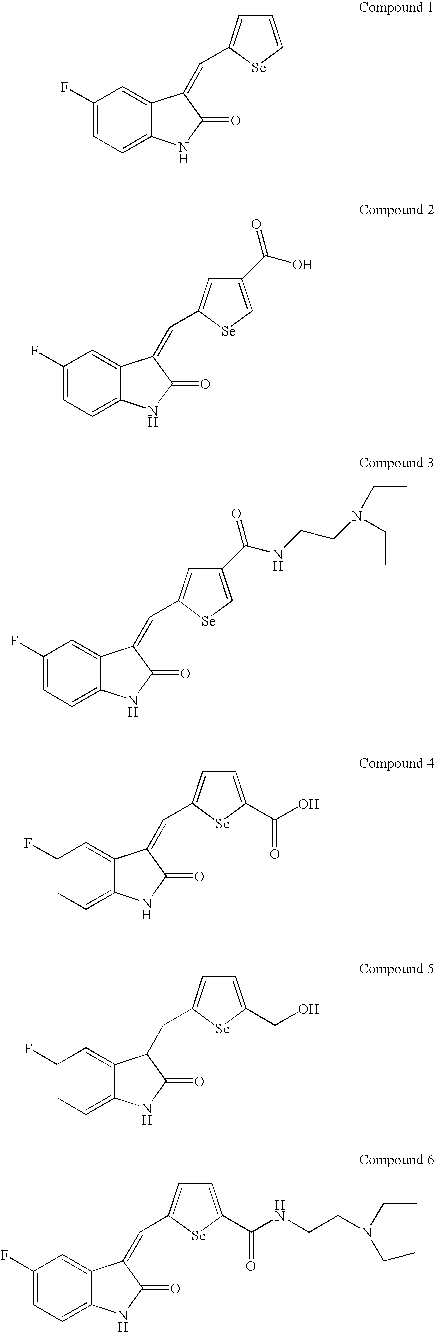

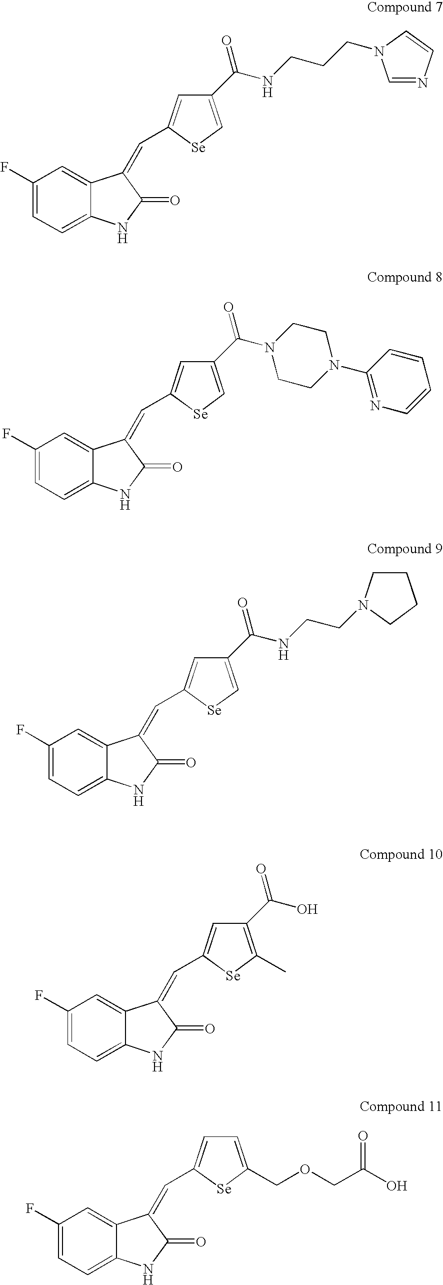

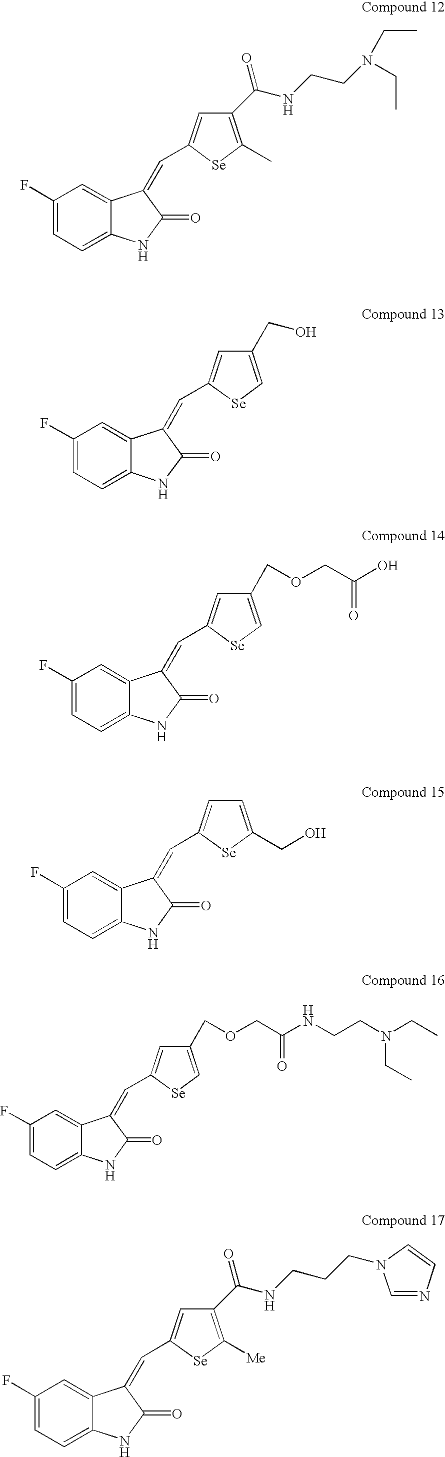

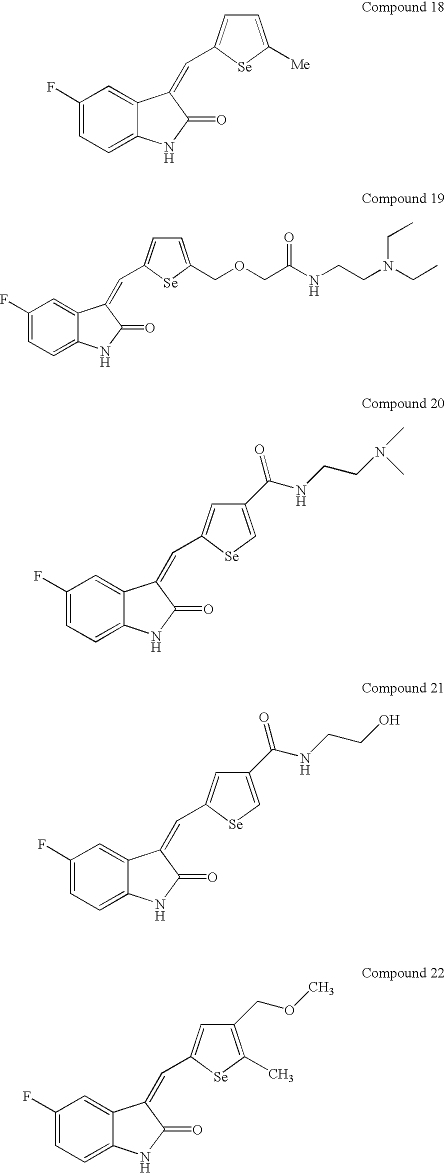

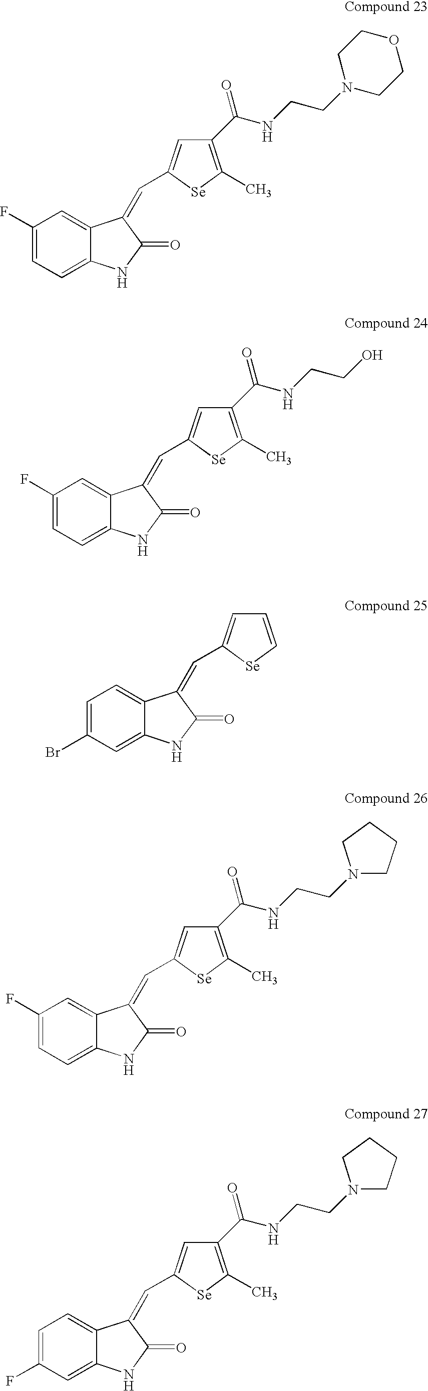

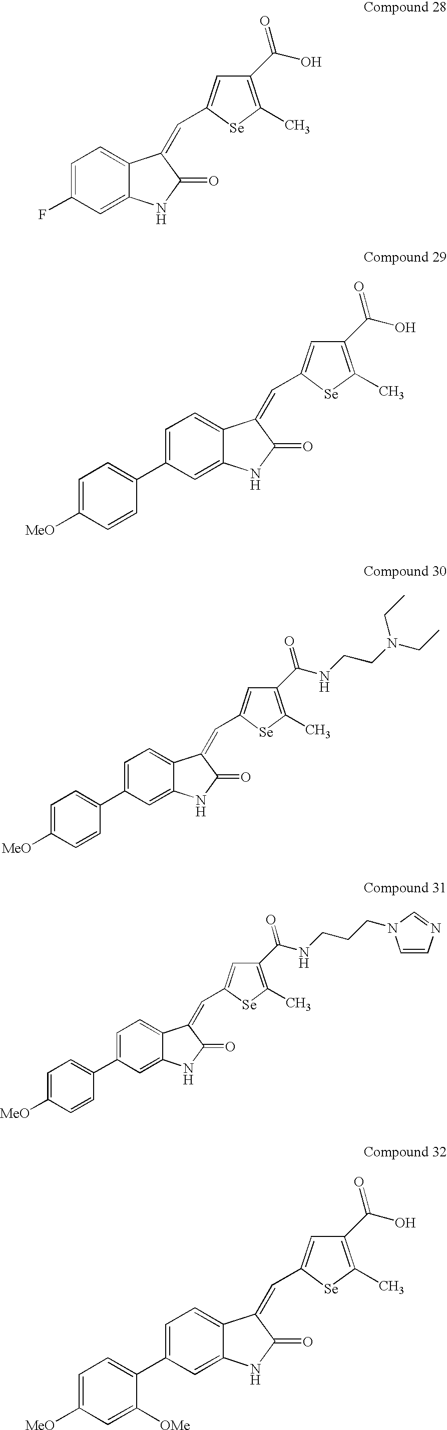

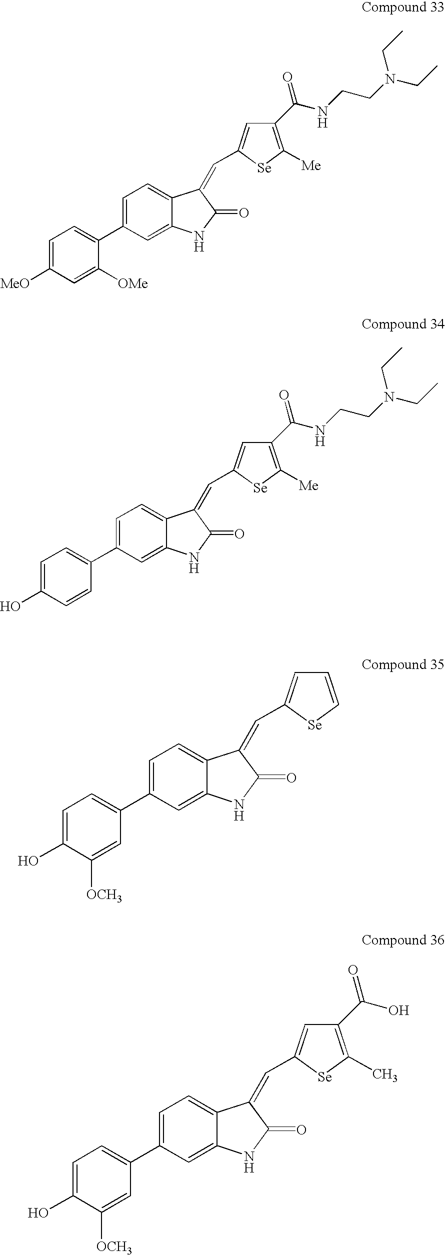

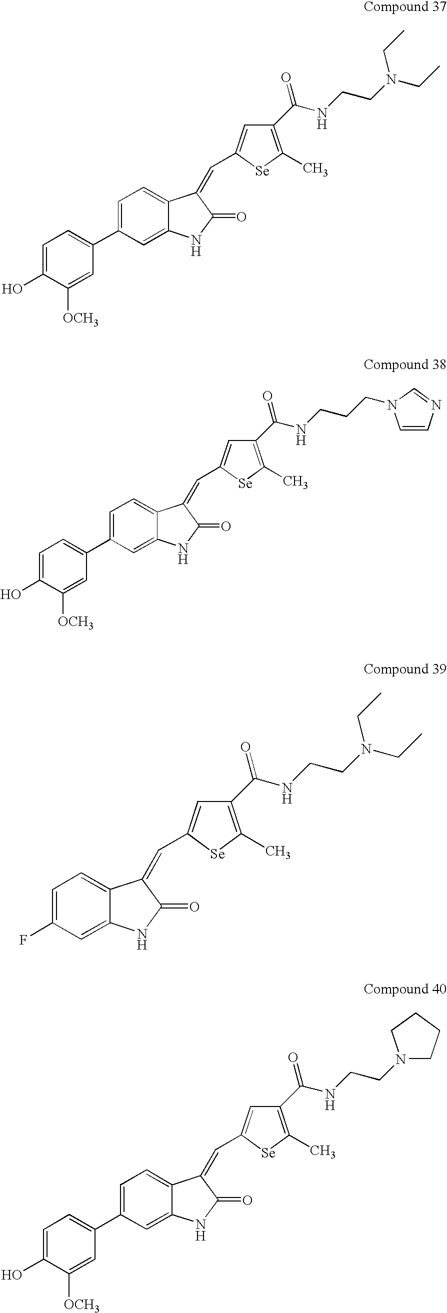

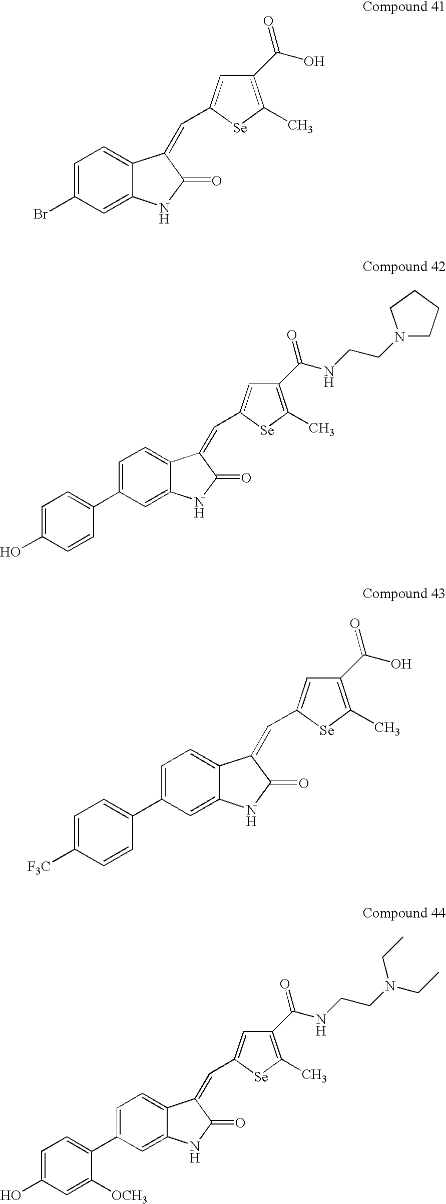

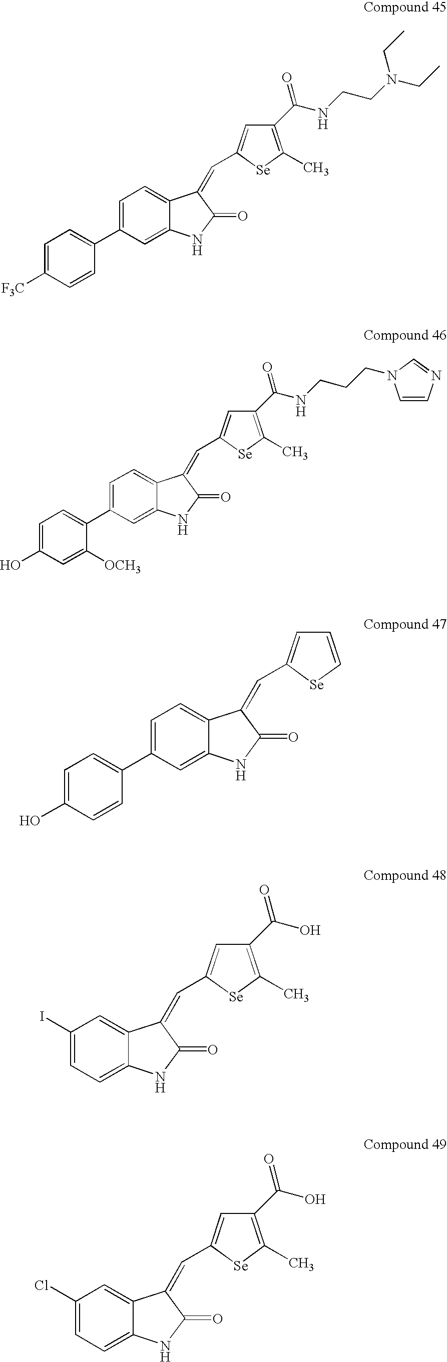

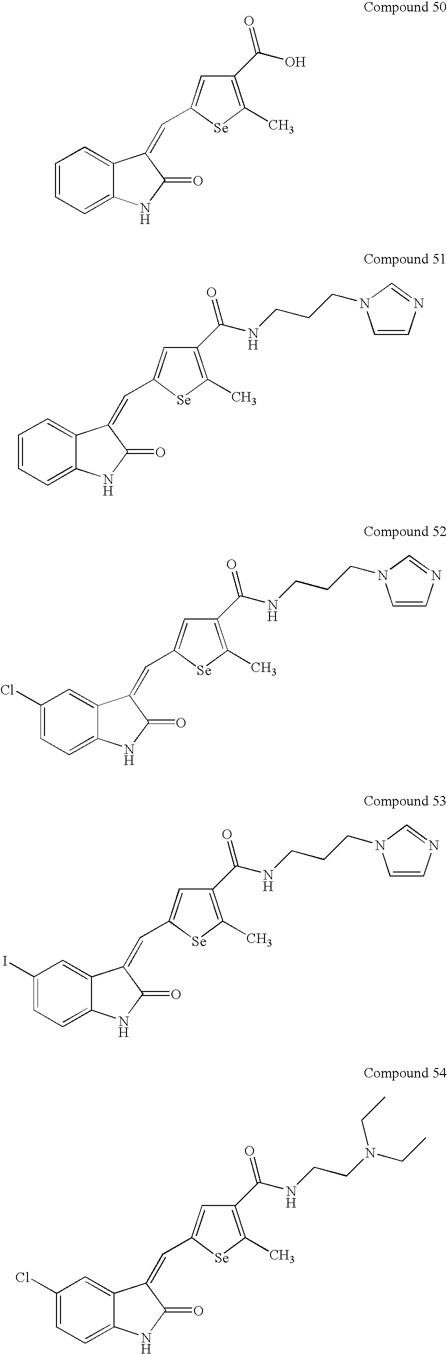

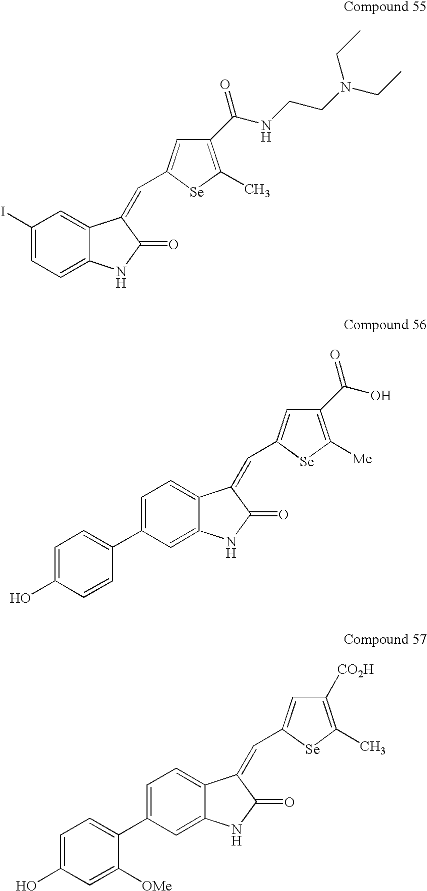

The selenophene compounds described above can be prepared by methods well known in the art, including the synthetic routes described below.

Scheme 1 shown below depicts an exemplary route for synthesizing selenophene compounds of this invention. Specifically, selenophene compound 3 is synthesized by coupling compound 1 (i.e., arylindolin-2-one) and compound 2 (i.e., selenophene-2-carbaldehyde) under basic conditions (e.g., piperidine/EtOH).

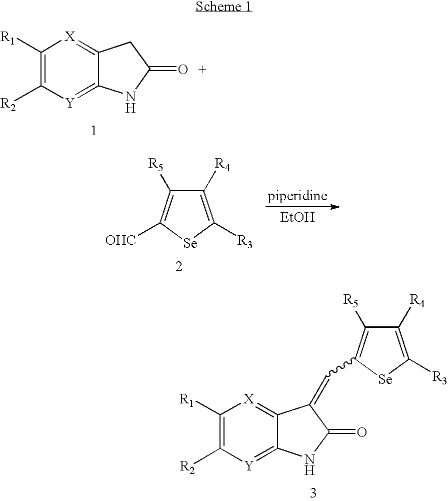

Scheme 2 shown below depicts an exemplary route for synthesizing compound 7 (i.e., arylindolin-2-ones) of this invention. Specifically, 2,5-dibromonitrobenzene 4 undergoes regioselective substitution with dimethyl malonate under basic conditions. The resulting malonate 5 is coupled with a boronic acid derivative to form the nitroaromatic compound 6, which is then transformed into the arylindolin-2-one 7.

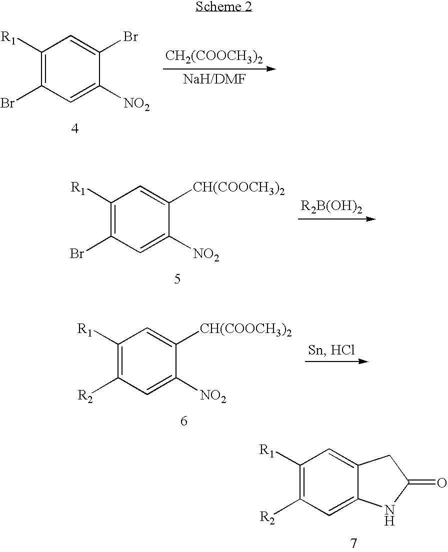

Scheme 3 shown below depicts two closely related routes for synthesizing compound 2 (selenophene-2-carbaldehydes). In the first example, a 2-substituted selenophene 8 is first reacted with dimethylformamide (DMF) in the presence of butylithium (BuLi) to form the selenophene-2-carbaldehyde 9. Compound 9 is then converted into the selenophene-2-carbaldehyde 10, following a regioselective bromination, protection of the aldehyde, lithium-bromine exchange, and quenching with an electrophile (E⁺). Compound 10 is further elaborated into required selenophene-2-carbaldehyde 11. In the latter example, the selenophene 12 without substitution at the 2 position is first reacted with dimethylformamide (DMF) in the presence of butylithium (BuLi) to form the selenophene-2-carbaldehyde 13. Compound 13 is then converted into the selenophene-2-carbaldehyde 14, following protection of the aldehyde, regioselective lithiation, and quenching with an electrophile (E⁺). Compound 14 is further elaborated into required selenophene-2-carbaldehyde 15.

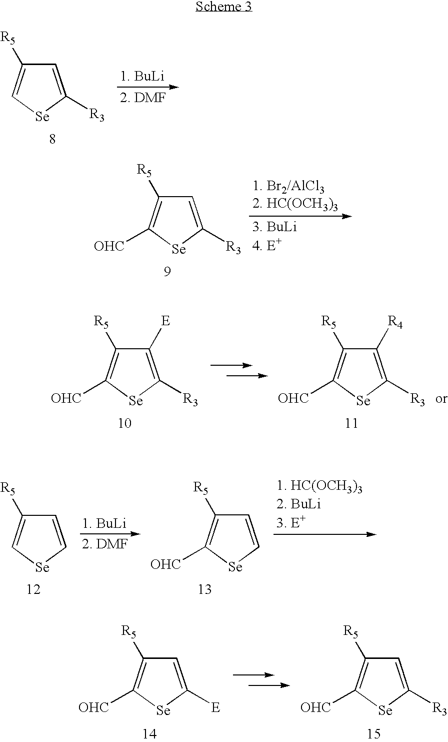

Examples 1-57 below provide detailed descriptions of the preparation of Compounds 1-57.

A selenophene compound synthesized above can be purified by a suitable method such as column chromatography, high-pressure liquid chromatography, or recrystallization.

Other selenophene compounds can be prepared using other suitable starting materials through the above synthetic routes and others known in the art. The methods described above may also additionally include steps, either before or after the steps described specifically herein, to add or remove suitable protecting groups in order to ultimately allow synthesis of the selenophene compounds. In addition, various synthetic steps may be performed in an alternate sequence or order to give the desired compounds. Synthetic chemistry transformations and protecting group methodologies (protection and deprotection) useful in synthesizing applicable selenophene compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The selenophene compounds mentioned herein may contain one or more asymmetric centers and a non-aromatic double bond. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition containing an effective amount of at least one of the selenophene compounds described above and a pharmaceutical acceptable carrier. A pharmaceutical acceptable carrier is a carrier compatible with the active ingredient of the composition (and preferably, capable of stabilizing the active ingredient) and not deleterious to the subject to be treated. One or more solubilizing agents can be utilized as pharmaceutical excipients for delivery of an active selenophene compound. Examples of other carriers include colloidal silicon oxide, magnesium stearate, cellulose, sodium lauryl sulfate, and D&C Yellow #10.

Further, this invention covers a method of administering an effective amount of one or more of the selenophene compounds to a patient having cancer. “An effective amount” refers to the amount of an active selenophene compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more selenophene compounds can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active selenophene compounds can also be administered in the form of suppositories for rectal administration.

The selenophene compounds described above can be preliminarily screened for their efficacy in inhibiting Chk1 kinase, VEGFR2, or PDGFR-Beta by an in vitro assay (See Example 58 below). The anti-cancer efficacy of those kinase inhibitors can then be confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1: (Z)-5-fluoro-3-(selenophen-2-ylmethylene)indolin-2-one

Selenophene-2-carbaldehyde was first prepared as follows. Butyllithium (115 ml, 1.6 M in hexane) was added dropwise to a stirred solution of 20.0 g of selenophene in 200 mL of dry THF under nitrogen at −78° C. The mixture was cooled with ice-water and stirred for 1 h. After cooling at −78° C., excessive DMF were added. The solution was allowed to reach room temperature. After 1 h, water was added to the mixture, the organic phase was separated. The ethereal phases were dried over magnesium sulfate and the ether was evaporated, followed by distillation under reduced pressure to give selenophene-2-carbaldehyde (16.6 g, 68%).

Selenophene-2-carbaldehyde was then reacted with 5-fluoroindolin-2-one as outlined in Scheme 1 to produce a mixture of Compound 1 and its olefin isomer, which was separated by column chromatography.

¹H NMR (500 MHz, DMSO-d₆) δ 10.68(s,1H), 8.64 (s,1H), 8.33(s,1H), 7.97(d,1H), 7.63(dd,1H), 7.50(t,1H), 7.04(m,1H), 6.86(dd,1H).

### EXAMPLE 2

### Preparation of Compound 2: (Z)-5-((5-fluoro-2-oxoindolin-3-ylidene)methyl)selenophene-3-carboxylic acid

5-Formylselenophene (10 g) was dissolved in anhydrous dichloromethane (200 mL) and the solution was cooled with ice-water. Aluminum chloride (25.22 g) was added with stirring. Bromine (3.5 mL) was added dropwise over a period of 30 min. After stirring at room temperature for 24 hours, the reaction mixture was poured onto crushed ice. The organic phase was separated and the aqueous phase was extracted with dichloromethane. The combined organic phases were dried over MgSO₄ and evaporated to give 6.75 g of crude prouct. A mixture of the residue (13.64 g), trimethyl orthoformate (10.64 mL), and anhydrous NH₄Cl (2.89 g) in MeOH (200 mL) was heated under reflux for overnight. After cooling to room temperature, the solution was distilled under reduced pressure. Ethyl acetate was added, and the organic solution was washed with water and brine, dried over MgSO₄, and evaporated. The crude product was distilled under reduced pressure to give 4-bromo-2-(dimethoxymethyl)selenophene (13.13 g, 85%).

A solution of 4-bromo-2-(dimethoxymethyl)selenophene (2.13 g) in 20 mL dry diethyl ether was added a 1.6 M solution of n-BuLi (4.9 mL) dropwise at −78° C. After being stirred at −78° C. for 30 min, the reaction mixture was treated with excess solid carbon monoxide. The solution was allowed to reach room temperature. After 1 h, the mixture was partitioned between 2N NaOH aqueous solution and diethyl ether. The aqueous phase was adjusted to pH 2 with 30% HCl aqueous solution and then extracted with ethyl acetate. The combined extracts were washed with water and brine, dried over MgSO₄, filtered, and concentrated to provide 5-formylselenophene-3-carboxylic acid (0.7 g, 50%).

5-formylselenophene-3-carboxylic acid was then reacted with 5-fluoroindolin-2-one as outlined in Scheme 1 to produce a mixture of Compound 2 and its olefin isomer, which was separated by column chromatography.

¹H NMR (500 MHz, DMSO-d₆) δ 10.78(s,1H), 9.28 (s,1H), 8.36(s,2H), 8.30(s,1H), 7.61(dd,1H), 7.07(m,1H), 6.87(dd,1H).

### EXAMPLE 3

### Preparation of Compound 3: (Z)-N-(2-(diethylamino)ethyl)-5-((5-fluoro-2-oxoindolin-3-ylidene)methyl)selenophene-3-carboxamide

Compound 3 was prepared in a manner similar to that described in Example 2.

¹H NMR (500 MHz, DMSO-d₆) δ 10.76(s,1H), 9.11 (s,1H), 8.32(m,3H), 7.65(dd,1H), 7.06(m,1H), 6.86(dd,1H), 3.29(m,4H), 2.54m,4H), 0.98(t,6H).

### EXAMPLE 4

### Preparation of Compound 4: (Z)-5-((5-fluoro-2-oxoindolin-3-ylidene)methyl)selenophene-2-carboxylic acid

A mixture of 5-formylselenophene (16.5 g), trimethyl orthoformate (20.5 mL), and anhydrous NH₄Cl (6.12 g) in MeOH (200 mL) was heated under reflux for overnight. After cooling to room temperature, the solution was distilled under reduced pressure. Ethyl acetate was added, and the organic solution was washed with water and brine, dried over MgSO₄, and evaporated to give crude product. The crude product was distilled under reduced pressure to give 2-(dimethoxymethyl)selenophene (12.7 g).

To a stirred solution of 2-(dimethoxymethyl)selenophene (2 g) in dry THF at −78° C. under nitrogen was added 8.6 mL of n-butyllithium (1.6 M in hexane) dropwise. The mixture was cooled with ice-water and stirred for 1 h. After cooling at −78° C., the reaction mixture was treated with excess solid carbon monoxide. The solution was allowed to reach room temperature. After 1 h, the mixture was partitioned between 2N NaOH aqueous solution and diethyl ether. The aqueous phase was adjusted to pH 2 with 30% HCl aqueous solution and then extracted with ethyl acetate. The combined extracts were washed with water and brine, dried over MgSO₄, filtered, and concentrated to provide 5-formylselenophene-2-carboxylic acid (1.1 g, 55%).

5-formylselenophene-2-carboxylic acid was then reacted with 5-fluoroindolin-2-one as outlined in Scheme 1 to produce a mixture of Compound 4 and its olefin isomer, which was separated by column chromatography.

¹H NMR (500 MHz, DMSO-d₆) δ 10.84(s,1H), 8.81 (s,1H), 8.33(s,2H), 8.01(d,1H), 7.94(d,1H), 7.67(t,1H), 7.10(m,1H), 6.88(dd,1H).

### EXAMPLE 5

### Preparation of Compound 5: 5-fluoro-3-((5-(hydroxymethyl)selenophen-2-yl)methyl)indolin-2-one

Compound 5 was prepared in a manner similar to that described in Example 15.

¹H NMR (500 MHz, DMSO-d₆) δ 9.37(br,1H), 7.20 (m,1H), 6.96(m,1H), 6.84(m,3H), 4.67(d,2H), 4.43(t,1H), 3.78(t,1H), 3.63(m,1H), 3.44(m,1H).

### EXAMPLE 6

### Preparation of Compound 6: (Z)-N-(2-(diethylamino)ethyl)-5-((5-fluoro-2-oxoindolin-3-ylidene)methyl)selenophene-2-carboxamide

Compound 6 was prepared in a manner similar to that described in Example 4.

¹H NMR (500 MHz, DMSO-d₆) δ 10.81(s,1H), 8.52 (t,1H), 8.23(s,1H), 7.96(d,1H), 7.93(d,1H), 7.65(dd,1H), 7.08(m,1H), 6.87(dd,1H), 3.30(m,4H), 2.55(m,4H), 0.98(t,6H).

### EXAMPLE 7

### Preparation of Compound 7: (Z)-N-(3-(1H-imidazol-1-yl)propyl)-5-((5-fluoro-2-oxoindolin-3-ylidene)methyl)selenophene-3-carboxamide

Compound 7 was prepared in a manner similar to that described in Example 2.

¹H NMR (500 MHz, DMSO-d₆) δ 10.76(s,1H), 9.15 (s,1H), 8.47(t,1H), 8.32(s,2H), 7.69(s,1H), 7.64(dd,1H), 7.23(s,1H), 7.07(m,1H), 6.92(s,1H), 6.87(dd,1H), 4.04(t,2H), 3.22(m,2H), 1.96(m,2H).

### EXAMPLE 8

### Preparation of Compound 8: (Z)-5-fluoro-3-((4-(4-(pyridin-2-yl)piperazine-1-carbonyl)selenophen-2-yl)methylene)indolin-2-one

Compound 8 was prepared in a manner similar to that described in Example 2.

¹H NMR (500 MHz, DMSO-d₆) δ 10.76(s,1H), 8.81 (s,1H), 8.41(d,2H), 8.34(s,1H), 8.06(s,1H), 7.61(dd,1H), 7.07(m,1H), 6.88(dd,1H), 6.68(d,1H), 3.83(br,4H), 3.65(br,4H).

### EXAMPLE 9

### Preparation of Compound 9: (Z)-5-((5-fluoro-2-oxoindolin-3-ylidene)methyl)-N-(2-(pyrrolidin-1-yl)ethyl)selenophene-3-carboxamide

Compound 9 was prepared in a manner similar to that described in Example 2.

¹H NMR (500 MHz, DMSO-d₆) δ 10.77 (s,1H), 9.16 (s,1H), 8.45(br,1H), 8.31(d,2H), 7.64(dd,1H), 7.06(m,1H), 6.87(dd,1H), 2.67(br,4H), 2.60(br,4H), 1.72(br,4H).

### EXAMPLE 10

### Preparation of Compound 10: (Z)-5-((5-fluoro-2-oxoindolin-3-ylidene)methyl)-2-methylselenophene-3-carboxylic acid

Compound 10 was prepared in a manner similar to that described in Example 2.

¹H NMR (500 MHz, DMSO-d₆) δ 10.73(s,1H), 8.26 (s,1H), 8.22(s,1H), 7.58(dd,1H), 7.04(m,1H), 6.85(dd,1H), 2.81(s,3H).

### EXAMPLE 11

### Preparation of Compound 11: (Z)-2-((5-((5-fluoro-2-oxoindolin-3-ylidene)methyl)selenophen-2-yl)methoxy)acetic acid

Excessive NaBH₄ was added to 5-(dimethoxymethyl)selenophene-2-carbaldehyde (1.1 g) in MeOH (20 mL) at 0° C. The solution was stirred for 1 h. Ethyl acetate was added, and the organic solution was washed with brine, dried over MgSO₄, and evaporated. The residue (1.2 g) and bromoacetic acid (1 g) was dissolved in dry DMF. The solution was cooled with ice-water and NaH (60%, 0.8 g) was added. The reaction mixture was stirred at 50° C. After 18 h, the mixture was adjusted to pH 2 with 30% HCl aqueous solution and then extracted with ethyl acetate. The combined extracts were washed with water and brine, dried over MgSO₄, filtered, and concentrated to provide 2-((5-formylselenophen-2-yl)methoxy)acetic acid (0.8 g).

2-((5-Formylselenophen-2-yl)methoxy)acetic acid was then reacted with 5-fluoroindolin-2-one as outlined in Scheme 1 to produce a mixture of Compound 11 and its olefin isomer, which was separated by column chromatography.

¹H NMR (500 MHz, DMSO-d₆) δ 10.70(s,1H), 8.26 (s,1H), 7.84(d,1H), 7.61(dd,1H), 7.34(d,1H), 7.03(m,1H), 6.85(dd,1H), 4.84(s,2H), 4.16(s,2H).

### EXAMPLE 12

### Preparation of Compound 12: (Z)-N-(2-(diethylamino)ethyl)-5-((5-fluoro-2-oxoindolin-3-ylidene)methyl)-2-methylselenophene-3-carboxamide

Compound 12 was prepared in a manner similar to that described in Example 2.

¹H NMR (500 MHz, DMSO-d₆) δ 10.70(s,1H), 8.17 (s,1H), 8.05(br,1H), 8.00(s,1H), 7.59(dd,1H), 7.02(m,1H), 6.84(dd,1H), 3.30(m,4H), 2.58(br,4H), 2.50(s,3H), 0.99(t,6H).

### EXAMPLE 13

### Preparation of Compound 13: (Z)-5-fluoro-3-((4-(hydroxymethyl)selenophen-2-yl)methylene)indolin-2-one

Excessive NaBH₄ was added to a solution of 5-(dimethoxymethyl)selenophene-3-carbaldehyde (1.1 g) in MeOH (20 mL) at 0° C. The solution was stirred for 1 h. Ethyl acetate was added, and the organic solution was washed with brine, dried over MgSO₄, and evaporated. The residue was dissolved in acetone (20 mL). Pyridium p-toluenesulfonate (cat.) was added and refluxed for 18 h. After cooling to room temperature, the solution was distilled under reduced pressure. Ethyl acetate was added, and the organic solution was washed with water and brine, dried over MgSO₄, and evaporated. The residue was purified by flash column chromatography to give 4-(hydroxymethyl)selenophene-2-carbaldehyde (0.55 g, 60%).

4-(hydroxymethyl)selenophene-2-carbaldehyde was then reacted with 5-fluoroindolin-2-one as outlined in Scheme 1 to produce a mixture of Compound 13 and its olefin isomer, which was separated by column chromatography.

¹H NMR (500 MHz, DMSO-d₆) δ 10.68(s,1H), 8.34 (s,1H), 8.28(s,1H), 7.93(s,1H), 7.63(dd,1H), 7.04(m,1H), 6.85(dd,1H), 5.26(t,1H), 4.48(d,2H).

### EXAMPLE 14

### Preparation of Compound 14: (Z)-2-((5-((5-fluoro-2-oxoindolin-3-ylidene)methyl)selenophen-3-yl)methoxy)acetic acid

Excessive NaBH₄ was added to 5-(dimethoxymethyl)selenophene-3-carbaldehyde (1.1 g) in MeOH (20 mL) at 0° C. The solution was stirred for 1 h. Ethyl acetate was added, and the organic solution was washed with brine, dried over MgSO₄, and evaporated. The residue (1.2 g) and bromoacetic acid (1 g) was dissolved in dry DMF. The solution was cooled with ice-water and NaH (60%, 0.8 g) was added. The reaction mixture was stirred at 50° C. After 18 h, the mixture was adjusted to pH 2 with 30% HCl aqueous solution and then extracted with ethyl acetate. The combined extracts were washed with water and brine, dried over MgSO₄, filtered, and concentrated to provide 2-((5-formylselenophen-3-yl)methoxy)acetic acid (0.8 g).

2-((5-Formylselenophen-3-yl)methoxy)acetic acid was then reacted with 5-fluoroindolin-2-one as outlined in Scheme 1 to produce a mixture of Compound 14 and its olefin isomer, which was separated by column chromatography.

¹H NMR (500 MHz, DMSO-d₆) δ 10.70(s,1H), 8.46 (t,1H), 8.31(s,1H), 7.96(s,1H), 7.64(m,1H), 7.05(m,1H), 6.85(dd,1H), 4.53(s,2H), 4.08(s,2H).

### EXAMPLE 15

### Preparation of Compound 15: (Z)-5-fluoro-3-((5-(hydroxymethyl)selenophen-2-yl)methylene)indolin-2-one

N-butyllithium (8.6 ml, 1.6 M in hexane) was added dropwise to a stirred solution of 2-(dimethoxymethyl)selenophene (2 g) in dry THF at −78° C. under nitrogen. The mixture was cooled with ice-water and stirred for 1 h. After cooling at −78° C., the reaction mixture was treated with excessive DMF. The solution was allowed to reach room temperature. After 1 h, ethyl acetate was added, and the organic solution was washed with saturated aqueous ammonium chloride and brine, dried over MgSO₄, and evaporated. The residue was purified by flash column chromatography to provide the 5-(dimethoxymethyl)selenophene-2-carbaldehyde (2 g, 90%).

To a solution of 5-(dimethoxymethyl)selenophene-2-carbaldehyde (1.1 g) in MeOH (20 mL), excessive NaBH₄ was added at 0° C. The solution was stirred for 1 h. Ethyl acetate was added, and the organic solution was washed with brine, dried over MgSO₄, and evaporated. The residue was dissolved in acetone (20 mL). Pyridium p-toluenesulfonate (cat.) was added and refluxed for 18 h. After cooling to room temperature, the solution was distilled under reduced pressure. Ethyl acetate was added, and the organic solution was washed with water and brine, dried over MgSO₄, and evaporated. The residue was purified by flash column chromatography to give 5-(hydroxymethyl)selenophene-2-carbaldehyde (0.55 g, 60% ).

5-(hydroxymethyl)selenophene-2-carbaldehyde was then reacted with 5-fluoroindolin-2-one as outlined in Scheme 1 to produce a mixture of Compound 15 and its olefin isomer, which was separated by column chromatography.

¹H NMR (500 MHz, DMSO-d₆) δ 10.66(s,1H), 8.23 (t,1H), 7.85(d,1H), 7.60(dd,1H), 7.24(d,1H), 7.02(m,1H), 6.84(dd,1H), 5.77(br,1H), 4.76(s,2H).

### EXAMPLE 16

### Preparation of Compound 16: (Z)-N-(2-(diethylamino)ethyl)-2-((5-((5-fluoro-2-oxoindolin-3-ylidene)methyl)selenophen-3-yl)methoxy)acetamide

Compound 16 was prepared in a manner similar to that described in Example 14.

¹H NMR (500 MHz, DMSO-d₆) δ 10.73(s,1H), 8.52 (s,1H), 8.29(d,1H), 7.98(s,1H), 7.82(s,1H), 7.61(dd,1H), 7.05(m,1H), 6.86(dd,1H), 4.54(s,2H), 3.92(s,2H), 3.26(br,4H), 2.66(br,4H), 1.02(t,6H).

### EXAMPLE 17

### Preparation of Compound 17: (Z)-N-(3-(1H-imidazol-1-yl)propyl)-5-((5-fluoro-2-oxoindolin-3-yl idene)methyl)-2-methylselenophene-3-carboxamide

Compound 17 was prepared in a manner similar to that described in Example 2.

¹H NMR (500 MHz, DMSO-d₆) δ 10.73(s,1H), 8.30 (m,1H), 8.20(s,1H), 7.68(s,1H), 7.60(dd,1H), 7.22(s,1H), 7.04(m,1H), 6.91(s,1H), 6.86(m,1H), 4.04(t,2H), 3.20(m,2H), 2.72(s,3H), 1.97(m,2H).

### EXAMPLE 18

### Preparation of Compound 18: (Z)-5-fluoro-3-((5-methylselenophen-2-yl)methylene)indolin-2-one

A stirred solution of selenophene (7 mL) in 100 mL dry THF was cooled at −78° C., and a 1.6 M solution of n-BuLi (61 mL) was added dropwise under nitrogen. The mixture was cooled with ice-water and stirred for 1 h. After cooling at −78° C., MeI (6.11 mL) was added dropwise. The cooling bath was removed and the mixture stirred for 2 h. The reaction mixture was cooled with ice-water and a 1.6 M solution of n-BuLi (61 mL) was added dropwise. The mixture was stirred for 30 min again. After cooling at −78° C., DMF were added. The solution was allowed to reach room temperature. After 1 h, ethyl acetate was added, and the organic solution was washed with saturated aqueous ammonium chloride and brine, dried over MgSO₄, and evaporated. The residue was purified by flash column chromatography (silica gel, EtOAc-hexane, 1:19) to give 11.8 g of 5-methylselenophene-2-carbaldehyde.

5-Methylselenophene-2-carbaldehyde was then reacted with 5-fluoroindolin-2-one as outlined in Scheme 1 to produce a mixture of Compound 18 and its olefin isomer, which was separated by column chromatography.

¹H NMR (500 MHz, DMSO-d₆) δ 10.65(s,1H), 8.21 (s,1H), 7.77(d,1H), 7.58(dd,1H), 7.15(d,1H), 7.01(m,1H), 6.83(m,2H), 2.61(s,3H).

### EXAMPLE 19

### Preparation of Compound 19: (Z)-N-(2-(diethylamino)ethyl)-2-((5-((5-fluoro-2-oxoindolin-3-ylidene)methyl)selenophen-2-yl)methoxy)acetamide

Compound 19 was prepared in a manner similar to that described in Example 11.

¹H NMR (500 MHz, DMSO-d₆) δ 10.73(s,1H), 8.28(s,1H), 7.86(d,1H), 7.62 (dd,1H), 7.38(d,1H), 7.04(m,1H), 6.85(dd,1H), 4.87(s,2H), 4.01(s,2H), 3.30(m,2H), 2.99(br,8H), 1.11(br,6H).

### EXAMPLE 20

### Preparation of Compound 20: (Z)-N-(2-(dimethylamino)ethyl)-5-((5-fluoro-2-oxoindolin-3-ylidene)methyl)selenophene-3-carboxamide

Compound 20 was prepared in a manner similar to that described in Example 2.

¹H NMR (500 MHz, DMSO-d₆) δ 10.75(s,1H), 9.14(s,1H), 8.32(m,3H), 7.64 (dd,1H), 7.06(m,1H), 6.87(dd,1H), 2.44(t,2H), 2.22(s,6H).

### EXAMPLE 21

### Preparation of Compound 21: (Z)-5-((5-fluoro-2-oxoindolin-3-ylidene)methyl)-N-(2-hydroxyethyl)selenophene-3-carboxamide

Compound 21 was prepared in a manner similar to that described in Example 2.

¹H NMR (500 MHz, DMSO-d₆) δ 10.74(s,1H), 9.16(s,1H), 8.38(br,1H), 8.32 (s,1H), 8.31(s,1H), 7.64(dd,1H), 7.06(m,1H), 6.87(dd,1H), 4.74(t,1H), 3.52(m,2H), 3.30(m,2H).

### EXAMPLE 22

### Preparation of Compound 22: (Z)-5-fluoro-3-((4-(methoxymethyl)-5-methylselenophen-2-yl)methylene)indolin-2-one

Compound 22 was prepared in a manner similar to that described in Example 2.

¹H NMR (500 MHz, DMSO-d₆) δ 10.64(s,1H), 8.20 (s,1H), 7.83(s,1H), 7.57(dd,1H), 7.01(m,1H), 6.83(dd,1H), 4.35(s,2H), 3.29(s,3H).

### EXAMPLE 23

### Preparation of Compound 23: (Z)-5-((5-fluoro-2-oxoindolin-3-ylidene)methyl)-2-methyl-N-(2-morpholinoethyl)selenophene-3-carboxamide

Compound 23 was prepared in a manner similar to that described in Example 2.

¹H NMR (500 MHz, DMSO-d₆) δ 10.71(s,1H), 8.19 (s,1H), 8.10(br,1H), 8.00 (s,1H), 7.60(dd,1H), 7.04(m,1H), 6.85(dd,1H), 3.59(t,4H), 2.72(s,3H), 2.47(m,8H).

### EXAMPLE 24

### Preparation of Compound 24: (Z)-5-((5-fluoro-2-oxoindolin-3-ylidene)methyl)-N-(2-hydroxyethyl)-2-methylselenophene-3-carboxamide

Compound 24 was prepared in a manner similar to that described in Example 2.

¹H NMR (500 MHz, DMSO-d₆) δ 10.71(s,1H), 8.18 (s,1H), 8.12(br,1H), 8.04 (s,1H), 7.60(dd,1H), 7.03(m,1H), 6.85(dd,1H), 4.72(t,1H), 3.51(dd,2H), 3.30(dd,2H), 2.72(s,3H).

### EXAMPLE 25

### Preparation of Compound 25: (Z)-6-bromo-3-(selenophen-2-ylmethylene)indolin-2-one

Compound 25 was prepared in a manner similar to that described in Example 1.

¹H NMR (500 MHz, DMSO-d₆) δ 10.82(s,1H), 8.64 (d,1H), 8.32 (t,1H), 8.00(d,1H), 7.67(d,1H), 7.49(dd,1H), 7.21(dd,1H), 7.03(d,1H).

### EXAMPLE 26

### Preparation of Compound 26: (Z)-5-((5-fluoro-2-oxoindolin-3-ylidene)methyl)-2-methyl-N-(2-(pyrrolidin-1-yl)ethyl)selenophene-3-carboxamide

Compound 26 was prepared in a manner similar to that described in Example 2.

¹H NMR (500 MHz, DMSO-d₆) δ 10.71(s,1H), 8.19 (s,2H), 8.13(br,1H), 8.01 (s,1H), 7.60(dd,1H), 7.03(m,1H), 6.84(dd,1H), 2.71(s,3H), 2.55(t,4H), 2.52(t,4H), 1.68(t,4H).

### EXAMPLE 27

### Preparation of Compound 27: (Z)-5-((6-fluoro-2-oxoindolin-3-ylidene)methyl)-2-methyl-N-(2-(pyrrolidin-1-yl)ethyl)selenophene-3-carboxamide

Compound 27 was prepared in a manner similar to that described in Example 2.

¹H NMR (500 MHz, DMSO-d₆) δ 10.86(s,1H), 8.10 (s,2H), 8.02 (s,1H), 7.70(dd,1H), 6.84(m,1H), 6.69(dd,1H), 2.70(s,3H), 2.56(t,4H), 2.52(t,4H), 1.69(t,4H).

### EXAMPLE 28

### Preparation of Compound 28: (Z)-5-((6-fluoro-2-oxoindolin-3-ylidene)methyl)-2-methylselenophene-3-carboxylic acid

Compound 28 was prepared in a manner similar to that described in Example 2.

¹H NMR (500 MHz, DMSO-d₆) δ 10.86(s,1H), 8.20 (s,1H), 8.17 (s,1H), 7.68(dd,1H), 6.83(m,1H), 6.68(dd,1H), 2.79(s,3H).

### EXAMPLE 29

### Preparation of Compound 29: (Z)-5-((6-(4-methoxyphenyl)-2-oxoindolin-3-ylidene)methyl)-2-methylselenophene-3-carboxylic acid

Sodium hydride (60%, 2.13 g) was added to a dry 100 mL flask under nitrogen. Anhydrous DMF (20 mL) was added, followed by dimethyl malonate (12 ml, 105 mmol). The reaction was heated briefly to 100° C. with stirring, and then cooled to room temperature. 2,5-dibromonitrobenzene (5.04 g, 17.9 mmol) was added and the reaction was heated at 100° C. for 3 hrs. After cooling to room temperature, ethyl acetate was added, and the organic solution was washed with saturated aqueous ammonium chloride and brine, dried over MgSO₄, and evaporated to give crude dimethyl 2-(4-bromo-2-nitrophenyl)malonate as a brown oil. The residue was purified by flash column chromatography to give 4.21 g (71%) of methyl (4-bromo-2-nitrophenyl) acetate as a yellow solid.

Pd(dppf)₂Cl₂—CH₂Cl₂ (0.25 g) was added to a mixture of 4-methoxyphenylboronic acid (1.5 g), dimethyl 2-(4-bromo-2-nitrophenyl)malonate (2 g) and 4 mL of 2M sodium carbonate solution in 8 mL of toluene and 8 mL of ethanol. The mixture was refluxed for 24 h, concentrated, and the residue extracted with ethyl acetate. The ethyl acetate layer was washed with water and brine, dried, and concentrated. The residue was purified by flash column chromatography to give dimethyl 2-(4′-methoxy-3-nitrobiphenyl-4-yl)malonate (1.8 g).

Hydrochloric acid (10 g) was added to a solution of dimethyl 2-(4′-methoxy-3-nitrobiphenyl-4-yl)malonate (3.5 g, mmol )in 20 mL EtOH. Then tin powder (5 g) was added and the reaction mixture was refluxed for 3 h. The solids were removed by filtration. The filtrate was concentrated and the residue extracted with ethyl acetate. The ethyl acetate layer was washed with water and brine, dried, and concentrated to give 6-(4-methoxyphenyl)indolin-2-one as a pale solid(0.85 g, 39%).

6-(4-methoxyphenyl)indolin-2-one was then reacted with 5-formyl-2-methylselenophene-3-carboxylic acid as outlined in Scheme 1 to produce a mixture of Compound 29 and its olefin isomer, which was separated by column chromatography.

¹H NMR (500 MHz, DMSO-d₆) δ 10.76(s,1H), 8.20 (s,1H), 8.16 (s,1H), 7.69(d,1H), 7.57(d,2H), 7.25(dd,1H), 7.03(s,1H), 7.00(d,2H), 3.77(s,3H), 2.78(s,3H).

### EXAMPLE 30

### Preparation of Compound 30: (Z)-N-(2-(diethylamino)ethyl)-5-((6-(4-methoxyphenyl)-2-oxoindolin-3-ylidene)methyl)-2-methylselenophene-3-carboxamide

Compound 30 was prepared in a manner similar to that described in Example 29.

¹H NMR (500 MHz, DMSO-d₆) δ 10.78(s,1H), 8.09(s,1H), 8.06 (br,1H), 8.02 (s,1H), 7.72(d,1H), 7.60(d,2H), 7.27(d,1H), 7.06(s,1H), 7.02(d,2H), 3.80(s,3H), 3.29(m,4H), 2.72(s,3H), 2.56(m,4H), 0.99(t,6H).

### EXAMPLE 31

### Preparation of Compound 31: (Z)-N-(3-(1H-imidazol-1-yl)propyl)-5-((6-(4-methoxyphenyl)-2-oxoindolin-3-ylidene)methyl)-2-methylselenophene-3-carboxamide

Compound 31 was prepared in a manner similar to that described in Example 29.

¹H NMR (500 MHz, DMSO-d₆) δ 10.78(s,1H), 8.27(m,1H), 8.11 (s,1H), 8.05 (s,1H), 7.72(d,1H), 7.69(s,1H), 7.60(d,2H), 7.28(dd,1H), 7.23(s,1H), 7.04(m,3H), 6.92(s,1H), 4.05(t,2H), 3.80(s,3H), 3.20(m,2H), 2.72(s,3H), 1.97(t,2H).

### EXAMPLE 32

### Preparation of Compound 32: (Z)-5-((6-(2,4-dimethoxyphenyl)-2-oxoindolin-3-ylidene)methyl)-2-methylselenophene-3-carboxylic acid

Compound 32 was prepared in a manner similar to that described in Example 29.

¹H NMR (500 MHz, DMSO-d₆) δ 10.68(s,1H), 8.22 (s,1H), 8.16(s,1H), 7.67(d,1H), 7.24(d,1H), 7.07(dd,1H), 6.97(d,1H), 6.67(d,1H), 6.62(dd,1H), 3.81(s,3H), 3.78(s,3H), 2.81(s,3H).

### EXAMPLE 33

### Preparation of Compound 33: (Z)-N-(2-(diethylamino)ethyl)-5-((6-(2,4-dimethoxyphenyl)-2-oxoindolin-3-ylidene)methyl)-2-methylselenophene-3-carboxamide

Compound 33 was prepared in a manner similar to that described in Example 29.

¹H NMR (500 MHz, DMSO-d₆) δ 10.68(s,1H), 8.08 (s,1H), 8.04 (br,1H), 8.01(s,1H), 7.67(d,1H), 7.25(d,1H), 7.07(dd,2H), 6.97(d,1H), 6.67(d,1H), 6.62(dd,1H), 3.81(s,3H), 3.78(s,3H), 3.28(m,4H), 2.72(s,3H), 2.52(m,4H), 0.98(t,6H).

### EXAMPLE 34

### Preparation of Compound 34: (Z)-N-(2-(diethylamino)ethyl)-5-((6-(4-hydroxyphenyl)-2-oxoindolin-3-ylidene)methyl)-2-methylselenophene-3-carboxamide

BBr₃ (2 g) was added to the solution of dimethyl 2-(4′-methoxy-3-nitrobiphenyl-4-yl)malonate (1.64 g) in anhydrous dichloromethane (10 mL). The mixture was stirred for 3 h at room temperature. Water was added, and the organic solution was washed with brine, dried over MgSO₄, and evaporated. The residue was purified by flash column chromatography to give dimethyl 2-(4′-hydroxy-3-nitrobiphenyl-4-yl)malonate as a pale yellow solid ( 1.5 g, 95% ).

Hydrochloric acid (3 g) was added to a solution of dimethyl 2-(4′-hydroxy-3-nitrobiphenyl-4-yl)malonate (1.5 g) in 10 mL EtOH. Then tin powder (2.5 g) was added and the reaction mixture was refluxed for 6 h. The solids were removed by filtration. The filtrate was concentrated and the residue extracted with ethyl acetate. The ethyl acetate layer was washed with water and brine, dried, and concentrated to give 6-(4-hydroxyphenyl)indolin-2-one as a powder (0.9 g).

6-(4-hydroxyphenyl)indolin-2-one was then reacted with 5-formyl-2-methylselenophene-3-carboxylic acid as outlined in Scheme 1 to produce a mixture of (Z)-5-((6-(4-Hydroxyphenyl)-2-oxoindolin-3-ylidene)methyl)-2-methylselenophene-3-carboxylic acid and its olefin isomer, which was separated by column chromatography.

Triethylamine (28 mg), 2-(diethylamino)ethylamine (27 mg), hydroxybenzotriazole (42 mg) and 1-(3-dimethylaminopropyl)-3-ethylcarbodiimided hydrochloride (53 mg) were added to a solution of (Z)-5-((6-(4-Hydroxyphenyl)-2-oxoindolin-3-ylidene)methyl)-2-methylselenophene-3-carboxylic acid (100 mg) in 3 mL DMF. The mixture was stirred for 18 h at room temperature. Ethyl acetate was added, and the organic solution was washed with water and brine, dried over MgSO₄, and evaporated to give Compound 34: (Z)-N-(2-(diethylamino)ethyl)-5-((6-(4-hydroxyphenyl)-2-oxoindolin-3-ylidene)methyl)-2-methylselenophene-3-carboxamide (85 mg) as a light orange solid.

¹H NMR (500 MHz, DMSO-d₆) δ 10.75(s,1H), 9.61(br,1H), 8.08 (s,1H), 8.04 (m,1H), 7.99(s,1H), 7.70(d,1H), 7.48(d,2H), 7.23(dd,1H), 7.01(d,1H), 6.85(d,2H), 3.28(m,4H), 2.54(m,4H), 0.98(t,6H).

### EXAMPLE 35

### Preparation of Compound 35: (Z)-6-(4-hydroxy-3-methoxyphenyl)-3-(selenophen-2-ylmethylene)indolin-2-one

Pd(dppf)₂Cl₂—CH₂Cl₂ (0.12 g) was added to a mixture of 2-methoxy-4-(4,4,5,5-tetramethyl-1,3,2-dioxaborolan-2-yl)phenol (0.9 g), dimethyl 2-(4-bromo-2-nitrophenyl)malonate (1 g) and 4 mL of 2M sodium carbonate solution in 12 mL DME. The mixture was refluxed for 24 h, concentrated, and the residue extracted with ethyl acetate. The ethyl acetate layer was washed with water and brine, dried, and concentrated to give a crude product. The residue was purified by flash column chromatography to give dimethyl 2-(4′-hydroxy-3′-methoxy-3-nitrobiphenyl-4-yl)malonate (0.5 g, 46%).

Hydrochloric acid (5.12 g) was added to a solution of dimethyl 2-(4′-hydroxy-3′-methoxy-3-nitrobiphenyl-4-yl)malonate (0.5 g)in 10 mL EtOH. Then tin powder (1.67 g) was added and the reaction mixture was refluxed for 3 h. The solids were removed by filtration. The filtrate was concentrated and the residue extracted with ethyl acetate. The ethyl acetate layer was washed with water and brine, dried, and concentrated to give 6-(4-Hydroxy-3-methoxyphenyl)indolin-2-one as a pale yellow solid (0.18 g, 49%).

6-(4-Hydroxy-3-methoxyphenyl)indolin-2-one was then reacted with selenophene-2-carbaldehyde as outlined in Scheme 1 to produce a mixture of Compound 35 and its olefin isomer, which was separated by column chromatography.

¹H NMR (500 MHz, DMSO-d₆) δ 10.70(s,1H), 9.17(s,1H), 8.57 (d,1H), 8.23 (s,1H), 7.96(d,1H), 7.73(d,1H), 7.47(m,1H), 7.27(d,1H), 7.18(s,1H), 7.07 (m,2H), 6.85 (d,1H), 3.86(s,3H).

### EXAMPLE 36

### Preparation of Compound 36: (Z)-5-((6-(4-hydroxy-3-methoxyphenyl)-2-oxoindolin-3-ylidene)methyl)-2-methylselenophene-3-carboxylic acid

Compound 36 was prepared in a manner similar to that described in Example 35.

¹H NMR (500 MHz, DMSO-d₆) δ 10.74(s,1H), 9.10(brs,1H), 8.21 (s,1H), 8.18 (s,1H), 7.70(d,1H), 7.18(s,1H), 7.07 (m,2H), 6.85 (d,1H), 3.86(s,3H), 2.80(s,2H).

### EXAMPLE 37

### Preparation of Compound 37: (Z)-N-(2-(diethylamino)ethyl)-5-((6-(4-hydroxy-3-methoxyphenyl)-2-oxoindolin-3-ylidene)methyl)-2-methylselenophene-3-carboxamide

Compound 37 was prepared in a manner similar to that described in Example 35.

¹H NMR (500 MHz, DMSO-d₆) δ 10.74(s,1H), 9.18(s,1H), 8.10 (s,1H), 8.05 (br,1H), 8.02(s,1H), 7.71(d,1H), 7.28(d,1H), 7.19(s,1H), 7.07(m,2H), 6.86(d,1H), 3.87(s,3H), 3.30(m,4H), 2.72(s,3H), 2.51(m,4H), 1.00(t,6H).

### EXAMPLE 38

### Preparation of Compound 38: (Z)-N-(3-(1H-imidazol-1-yl)propyl)-5-((6-(4-hydroxy-3-methoxyphenyl)-2-oxoindolin-3-ylidene)methyl)-2-methylselenophene-3-carboxamide

Compound 38 was prepared in a manner similar to that described in Example 35.

¹H NMR (500 MHz, DMSO-d₆) δ 10.75(s,1H), 9.19(s,1H), 8.26 (m,1H), 8.10 (s,1H), 8.05(s,1H), 7.71(d,1H), 7.68(s,1H), 7.29(d,1H), 7.23(s,1H), 7.18(d,1H), 7.08(m,2H), 6.92(s,1H), 6.86(d,1H), 4.05(t,2H), 3.87(s,3H), 3.20(m,2H), 1.96(m,2H).

### EXAMPLE 39

### Preparation of Compound 39: (Z)-N-(2-(diethylamino)ethyl)-5-((6-fluoro-2-oxoindolin-3-ylidene)methyl)-2-methylselenophene-3-carboxamide.

Compound 39 was prepared in a manner similar to that described in Example 2

¹H NMR (500 MHz, DMSO-d₆) δ 10.88(s,1H), 8.10(s,1H), 8.04 (m,lH), 8.00 (s,1H), 7.71(dd,1H), 6.78(m,1H), 6.70(dd,1H), 3.32(m,4H), 2.71(s,3H), 2.54(m,4H), 0.97(t,6H).

### EXAMPLE 40

### Preparation of Compound 40: 1(Z)-5-((6-(4-hydroxy-3-methoxyphenyl)-2-oxoindolin-3-ylidene)methyl)-2-methyl-N-(2-(pyrrolidin-1-yl)ethyl)selenophene-3-carboxamide

Compound 40 was prepared in a manner similar to that described in Example 35.

¹H NMR (500 MHz, DMSO-d₆) δ 10.73(s,1H), 9.20(br,1H), 8.10(br,2H), 8.02 (s,1H), 7.70(d,1H), 7.18(d,1H), 7.06(m,2H), 6.86(d,1H), 3.86(s,3H),3.33(m,4H), 2.71(s,3H), 2.57(m,4H), 1.70(m,4H).

### EXAMPLE 41

### Preparation of Compound 41: (Z)-5-((6-bromo-2-oxoindolin-3-ylidene)methyl)-2-methylselenophene-3-carboxylic acid

Compound 41 was prepared in a manner similar to that described in Example 2.

¹H NMR (500 MHz, DMSO-d₆) δ 10.84(s,1H), 8.23(s,2H), 7.62 (d,1H), 7.19 (m,1H), 7.00(s,1H), 2.80(s,3H).

### EXAMPLE 42

### Preparation of Compound 42: (Z)-5-((6-(4-hydroxyphenyl)-2-oxoindolin-3-ylidene)methyl)-2-methyl-N-(2-(pyrrolidin-1-yl)ethyl)selenophene-3-carboxamide

Compound 42 was prepared in a manner similar to that described in Example 34.

¹H NMR (500 MHz, DMSO-d₆) δ 10.74(s,1H), 9.61(br,1H), 8.11 (br,1H), 8.07 (s,1H), 8.01(s,1H), 7.69(d,1H), 7.47(d,2H), 7.23(dd,1H), 7.01(d,1H), 6.84(d,2H), 2.70(s,3H), 2.60(m,4H), 2.52(m,4H), 1.69(m,4H).

### EXAMPLE 43

### Preparation of Compound 43: (Z)-2-methyl-5-((2-oxo-6-(4-(trifluoromethyl)phenyl)indolin-3-ylidene)methyl)selenophene-3-carboxylic acid

Compound 43 was prepared in a manner similar to that described in Example 29.

¹H NMR (500 MHz, DMSO-d₆) δ 10.88(s,1H), 8.26 (d,2H), 7.88(d,2H), 7.80(m,3H), 7.40(dd,1H), 7.15(d,1H), 2.81(s,3H).

### EXAMPLE 44

### Preparation of Compound 44: (Z)-N-(2-(diethylamino)ethyl)-5-((6-(4-hydroxy-2-methoxyphenyl)-2-oxoindolin-3-ylidene)methyl)-2-methylselenophene-3-carboxamide

Compound 44 was prepared in a manner similar to that described in Example 35.

¹H NMR (500 MHz, DMSO-d₆) δ 10.62(s,1H), 9.60(br,1H), 8.03 (s,1H), 8.01 (br,1H), 8.00(s,1H), 7.62(d,1H), 7.09(d,1H), 7.02(m,1H), 6.92(s,1H), 6.49(d,1H), 6.42(dd,1H), 3.71(s,3H), 3.26(m,4H), 2.69(s,3H), 2.52(m,4H), 0.96(m,6H).

### EXAMPLE 45

### Preparation of Compound 45: (Z)-N-(2-(diethylamino)ethyl)-2-methyl-5-((2-oxo-6-(4-(trifluoromethyl)phenyl)indolin-3-ylidene)methyl)selenophene-3-carboxamide

Compound 45 was prepared in a manner similar to that described in Example 29.

¹H NMR (500 MHz, DMSO-d₆) δ 10.87(s,1H), 8.18 (s,1H), 8.05 (d,2H), 7.89(d,2H), 7.81(m,3H), 7.39(dd,1H), 7.16(dd,1H), 3.28(m,4H), 2.71(s,3H), 2.54(m,4H), 0.98(t,6H).

### EXAMPLE 46

### Preparation of Compound 46: (Z)-N-(3-(1H-imidazol-1-yl)propyl)-5-((6-(4-hydroxy-2-methoxyphenyl)-2-oxoindolin-3-ylidene)methyl)-2-methylselenophene-3-carboxamide

Compound 46 was prepared in a manner similar to that described in Example 35.

¹H NMR (500 MHz, DMSO-d₆) δ 10.65(s,1H), 9.61 (s,1H), 8.25 (m,1H), 8.05(s,1H), 8.03(s,1H), 7.65(m,2H), 7.22(s,1H), 7.11(d,1H), 7.04(dd,1H), 6.94(d,1H), 6.90(s,1H), 6.50(d,1H), 6.44(dd,1H), 4.04(t,2H), 3.72(s,3H), 3.18(m,2H), 2.71(s,3H), 1.94(t,2H).

### EXAMPLE 47

### Preparation of Compound 47: (Z)-6-(4-hydroxyphenyl)-3-(selenophen-2-ylmethylene)indolin-2-one

A reaction mixture of 5-formylselenophene (110 mg), 6-(4-hydroxyphenyl)indolin-2-one (150 mg), and piperidine (20 mg) in 5 mL of ethanol was heated at 90° C. for 18 h and cooled to room temperature. Acetic acid (2 mL) was slowly added. The precipitate was filtered, washed with water and cold ethanol, and dried to give (Z)-6-(4-Hydroxyphenyl)-3-(selenophen-2-ylmethylene)indolin-2-one (190 mg) as an orange solid.

¹H NMR (500 MHz, DMSO-d₆) δ 10.73(s,1H), 9.60(s,1H), 8.58 (d,1H), 8.23 (s,1H), 7.96(d,1H), 7.73(d,1H), 7.49(m,3H), 7.24(m,1H), 7.03(d,1H), 6.86(d,2H).

### EXAMPLE 48

### Preparation of Compound 48: (Z)-5-((5-iodo-2-oxoindolin-3-ylidene)methyl)-2-methylselenophene-3-carboxylic acid

Compound 48 was prepared in a manner similar to that described in Example 2.

¹H NMR (500 MHz, DMSO-d₆) δ 10.81(s,1H), 8.30 (s,lH), 8.22(s,1H), 8.06(s,1H), 7.51(dd,1H), 6.71(d,1H), 2.80(s,3H).

### EXAMPLE 49

### Preparation of Compound 49: (Z)-5-((5-chloro-2-oxoindolin-3-ylidene)methyl)-2-methylselenophene-3-carboxylic acid

Compound 49 was prepared in a manner similar to that described in Example 2.

¹H NMR (500 MHz, DMSO-d₆) δ 10.85(s,1H), 8.32 (s,1H), 8.23(s,1H), 7.80(d,1H), 7.24(dd,1H), 6.87(d,1H), 2.81(s,3H).

### EXAMPLE 50

### Preparation of Compound 50: (Z)-2-methyl-5-((2-oxoindolin-3-ylidene)methyl)selenophene-3-carboxylic acid

Compound 50 was prepared in a manner similar to that described in Example 2.

¹H NMR (500 MHz, DMSO-d₆) δ 10.71(s,1H), 8.22 (s,1H), 8.18(s,1H), 7.67(d,1H), 7.20(m,1H), 7.00(m,1H), 6.86(d,1H), 2.80(s,3H).

### EXAMPLE 51

### Preparation of Compound 51: (Z)-N-(3-(1H-imidazol-1-yl)propyl)-2-methyl-5-((2-oxoindolin-3-ylidene)methyl)selenophene-3-carboxamide

Compound 51 was prepared in a manner similar to that described in Example 2.

¹H NMR (500 MHz, DMSO-d₆) δ 10.71(s,1H), 8.26(t,1H), 8.10 (s,1H), 8.04(s,1H), 7.68(m,2H), 7.21(m,2H), 7.01(m,1H), 6.99(s,1H), 6.88(m,1H), 4.04(t,2H), 3.18(m,2H), 2.70(s,3H), 1.94(m,2H).

### EXAMPLE 52

### Preparation of Compound 52: (Z)-N-(3-(1H-imidazol-1-yl)propyl)-5-((5-chloro-2-oxoindolin-3-ylidene)methyl)-2-methylselenophene-3-carboxamide

Compound 52 was prepared in a manner similar to that described in Example 2.

¹H NMR (500 MHz, DMSO-d₆) δ 10.84(s,1H), 8.30 (s,1H), 8.25(s,1H), 8.03(s,1H), 7.80(s,1H), 7.77(s,1H), 7.23(m,2H), 6.90(s,1H), 6.87(d,1H), 4.03(t,2H), 3.18(m,2H), 2.72(s,3H), 1.94(m,2H).

### EXAMPLE 53

### Preparation of Compound 53: (Z)-N-(3-(1H-imidazol-1-yl)propyl)-5-((5-iodo-2-oxoindolin-3-ylidene)methyl)-2-methylselenophene-3-carboxamide

Compound 53 was prepared in a manner similar to that described in Example 2.

¹H NMR (500 MHz, DMSO-d₆) δ 10.82(s,1H), 8.29 (t,1H), 8.25(s,1H), 8.06(s,1H), 8.02(s,1H), 7.70(s,1H), 7.51(d,1H), 7.24(s,2H), 6.92(s,1H), 6.71(d,1H), 4.04(t,2H), 3.18(m,2H), 2.70(s,3H), 1.94(m,2H).

### EXAMPLE 54

### Preparation of Compound 54: (Z)-5-((5-chloro-2-oxoindolin-3-ylidene)methyl)-N-(2-(diethylamino)ethyl)-2-methylselenophene-3-carboxamide

Compound 54 was prepared in a manner similar to that described in Example 2.

¹H NMR (500 MHz, DMSO-d₆) δ 10.83(s,1H), 8.25 (s,1H), 8.07(t,1H), 7.99(s,1H), 7.82(d,1H), 7.23(dd,1H), 6.86(d,1H), 3.27(m,4H), 2.70(s,3H), 2.54(m,4H), 0.97(t,6H).

### EXAMPLE 55

### Preparation of Compound 55: (Z)-N-(2-(diethylamino)ethyl)-5-((5-iodo-2-oxoindolin-3-ylidene)methyl)-2-methylselenophene-3-carboxamide

Compound 55 was prepared in a manner similar to that described in Example 2.

¹H NMR (500 MHz, DMSO-d₆) δ 10.81(s,1H), 8.24 (s,1H), 8.07(s,1H), 8.04(br,1H), 7.98(s,1H), 7.51(d,1H), 6.71(d,1H), 3.27(m,4H), 2.70(s,3H), 2.54(m,4H), 0.97(t,6H).

### EXAMPLE 56

### Preparation of Compound 56: (Z)-5-((6-(4-hydroxyphenyl)-2-oxoindolin-3-ylidene)methyl)-2-methylselenophene-3-carboxylic acid

Compound 56 was prepared in a manner similar to that described in Example 34.

¹H NMR (500 MHz, DMSO-d₆) δ 10.76(s,1H), 9.62(br,1H), 8.21(s,1H), 8.17 (s,1H), 7.70(t,2H), 7.23(t,1H), 7.02(d,1H), 6.86(d,2H), 2.81(s,3H).

### EXAMPLE 57

### Preparation of Compound 57: (Z)-5-((6-(4-hydroxy-2-methoxyphenyl)-2-oxoindolin-3-ylidene)methyl)-2-methylselenophene-3-carboxylic acid

Compound 57 was prepared in a manner similar to that described in Example 34.

¹H NMR (500 MHz, DMSO-d₆) δ 10.65(s,1H), 9.62(br,1H), 8.20 (s,1H), 8.14 (s,1H), 7.64(d,1H), 7.11(d,1H), 7.04(dd,1H), 6.94(s,1H), 6.51(d,1H), 6.44(dd,1H), 3.72(s,3H), 2.80(s,3H).

### EXAMPLE 58

### Assay for Kinase Activities

The kinase activities of Chk1, vascular endothelial growth factor receptor 2 (VEGFR2), and Platelet-derived growth factor receptor-Beta (PDGFR-Beta) were determined as follows. In the presence or absence of a test compound, 10 ng of a purified catalytic subunit of Chk1, VEGFR2, or PDGFR-Beta were incubated with 5 ng of a phosphorylate substrate CHKtide KKKVSRS-GLYRSPSMPENLNRPR or poly(Glu:Tyr) (4:1) (Sigma), and radiolabeled ATP-P₃₃ (50-100 μM) in a reaction buffer (pH 7.0) containing 8 mM 3-(N-morpholino)propanesulfonic acid (MOPS) and 0.2 mM ethylenediaminetetraacetic acid (EDTA) at 30° C. for 30 minutes. Phorphoric acid (3%) was added to quench the reaction and the reaction mixture was poured through a Unifilter-96 GF/B filter plate (PerkinElmer). After extensive wash with distilled water, the filter plate was air dried and then placed in Scintillation Ready Safe Cocktail (Beckman). The radioactivity of the plate, indicating the level of kinase activity, was determined by a TopCount (PerkinElmer) microplate reader.

Compounds 1-19 and 25-57 were tested for their efficacy in inhibiting kinase activities of Chk1, VEGFR2, and PDGFR-Beta following the method described above. Unexpectedly, among them, 25 compounds inhibited Chk1 activity at IC₅₀ values between about 0.3 nM to about 600 nM; 5 compounds inhibited Chk1 activity at IC₅₀ values between about 0.3 nM to about 0.5 nM; 29 compounds inhibited VEGFR2 activity at IC₅₀ values between about 8 nM to about 400 nM; and 7 compounds inhibited PDGFR-Beta at IC₅₀ values between about 30 nM to about 150 nM.

Moreover, the test compounds covered by formula (I) showed good inhibition activity against various kinases, e.g., Ca²⁺/Calmodulin-dependent kinase, Minibrain kinase/dual-specificity tyrosine phosphorylation-regulated kinase (Mnb/Dyrk1a), MAP/microtubule affinity-regulating kinase 1(MARK1), 3-phosphoinositide-dependent protein kinase 1 (PDK1), PIM1, Protein Kinase D2 (PKD2), testis-specific serine kinase 2 (TSSK2), vascular endothelial growth factor receptor 1 (VEGFR-1), FMS-like tyrosine kinase-3 (FLT3), anaplastic lymphoma kinase (ALK), RET, and Nima-related kinase 2 (NEK2).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 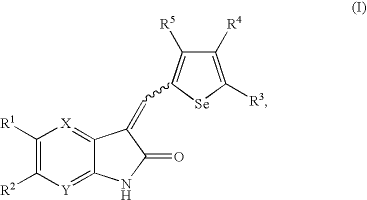
wherein
each of X and Y, independently, is N or CR′, in which R′ is H, halo, alkyl, cycloalkyl, aryl, heteroaryl, heterocycloalkyl, OH, alkoxyl, aryloxyl, —C(O)R^{a}, —NR^{b}R^{c}, —C(O)NR^{b}R^{c}, —(CH₂)ₙC(O)R^{a}, —(CH₂)ₙNR^{b}R^{c}, or —(CH₂)ₙC(O)NR^{b}R^{c}, in which R^{a} is H, OH, alkoxyl, or aryloxyl, each of R^{b} and R^{c}, independently, is H, alkyl, cycloalkyl, aryl, or heteroaryl, or R^{b} and R^{c} together with the N atom to which they are attached are heterocycloalkyl, and n is 1, 2, 3, or 4;
each of R¹ and R², independently, is H, halo, alkyl, OH, alkoxyl, aryl, heteroaryl, CN, —NR^{a1}R^{b1}, —NR^{a1}C(O)R^{b1}, —C(O)R^{c1}, —NR^{a1}S(O)₂R^{b1}, —S(O)₂NR^{a1}R^{b1}, or —SO₂R^{d1}, in which each of R^{a1} and R^{b1}, independently, is H, alkyl, cycloalkyl, heterocycloalkyl, aryl, or heteroaryl, or R^{a1} and R^{b1} together with the N atom or the N and C atoms or the N and S atoms to which they are attached are heterocycloalkyl, R^{c1} is H, OH, alkoxyl, or aryloxyl, and R^{d1} is alkyl, aryl, or heteroaryl; and
each of R³, R⁴, and R⁵, independently, is H, halo, alkyl, OH, alkoxyl, aryloxyl, —C(O)R^{a2}, or —C(O)NR^{b2}R^{c2}, in which R^{a2} is OH, alkoxyl, aryloxyl, or heterocycloalkyl, and each of R^{b2} and R^{c2}, independently, is H, alkyl, cycloalkyl, aryl or heteroaryl, or R^{b2} and R^{c2} together with the N atom to which they are attached are heterocycloalkyl, or R³ and R⁴ together with the C atoms to which they are attached are cycloalkyl or heterocycloalkyl, or R⁴ and R⁵ together with the C atoms to which they are attached are cycloalkyl or heterocycloalkyl.

2. The compound of claim 1, wherein formula (I) is

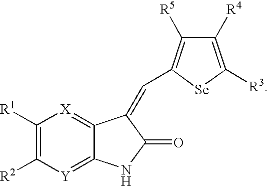

3. The compound of claim 2, wherein each of X and Y is CH.

4. The compound of claim 3, wherein R¹ is H and R² is phenyl optionally substituted with OH, alkoxyl, aryloxyl, or alkyl.

5. The compound of claim 4, wherein R² is 4-hydroxyphenyl optionally substituted with OCH₃.

6. The compound of claim 5, wherein each of R³, R⁴, and R⁵, independently, is H, CH₃, —COOH, —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}, in which m is 2 or 3, R^{a3} is H, OH, alkyl, alkoxyl, aryloxyl, or heterocycloalkyl, and each of R^{b3} and R^{c3}, independently, is H, alkyl, cycloalkyl, aryl, or heteroaryl, or R^{b2} and R^{c2} together with the N atom to which they are attached are heterocycloalkyl or heteroaryl.

7. The compound of claim 6, wherein at least one of R³, R⁴, and R⁵ is H and at least one of the others is —COOH or —C(O)NH(CH₂)ₘNR^{b3}R^{c3}.

8. The compound of claim 4, wherein R² is 4-methoxyphenyl optionally substituted with OCH₃.

9. The compound of claim 8, wherein each of R³, R⁴, and R⁵, independently, is H, CH₃, —COOH, —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}, in which m is 2 or 3, R^{a3} is H, OH, alkyl, alkoxyl, aryloxyl, or heterocycloalkyl, and each of R^{b3} and R^{c3}, independently, is H, alkyl, cycloalkyl, aryl, or heteroaryl, or R^{b2} and R^{c2} together with the N atom to which they are attached are heterocycloalkyl or heteroaryl.

10. The compound of claim 9, wherein at least one of R³, R⁴, and R⁵ is H and at least one of the others is —COOH or —C(O)NH(CH₂)ₘNR^{b3}R^{c3}.

11. The compound of claim 3, wherein one of R¹ and R² is halo and the other is H.

12. The compound of claim 11, wherein halo is F.

13. The compound of claim 12, wherein each of R³, R⁴, and R⁵, independently, is H, alkyl optionally substituted with OH or OCH₃, —COOH, —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}, in which m is 2 or 3, R^{a3} is H, OH, alkyl, alkoxyl, aryloxyl, or heterocycloalkyl, and each of R^{b3} and R^{c3}, independently, is H, alkyl, cycloalkyl, aryl or heteroaryl, or R^{b2} and R^{c2} together with the N atom to which they are attached are heterocycloalkyl or heteroaryl.

14. The compound of claim 13, wherein at least one of R³, R⁴, and R⁵ is H and at least one of the others of is —COOH , —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}.

15. The compound of claim 11, wherein each of R³, R⁴, and R⁵, independently, is H, alkyl optionally substituted with OH or OCH₃, —COOH, —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}, in which m is 2 or 3, R^{a3} is H, OH, alkyl, alkoxyl, aryloxyl, or heterocycloalkyl, and each of R^{b3} and R^{c3} , independently, is H, alkyl, cycloalkyl, aryl, or heteroaryl, or R^{b2} and R^{c2} together with the N atom to which they are attached are heterocycloalkyl or heteroaryl.

16. The compound of claim 15, wherein at least one of R³, R⁴, and R⁵ is H and at least one of the others is —COOH , —CH₂OCH₂C(O)R^{a3}, —C(O)NH(CH₂)ₘNR^{b3}R^{c3}, or —CH₂OCH₂C(O)NH(CH₂)ₘNR^{b3}R^{c3}.

17. The compound of claim 1, wherein each of X and Y is CH.

18. The compound of claim 17, wherein R¹ is H and R² is phenyl optionally substituted with OH, alkoxyl, aryloxyl, or alkyl.

19. The compound of claim 17, wherein one of R¹ and R² is halo and the other is H.

20. The compound of claim 1, wherein R¹ is H and R² is phenyl optionally substituted with OH, alkoxyl, aryloxyl, or alkyl.

21. The compound of claim 1, wherein one of R¹ and R² is halo and the other is H.

22. The compound of claim 1, wherein at least one of X and Y is N.

23. The compound of claim 1, wherein the compound is one of Compounds 1-57.

24. A pharmaceutical composition, comprising the compound of claim 1 and a pharmaceutically acceptable carrier.

25. A method of treating cancer, comprising administering to a subject in need thereof an effective amount of the compound of claim 1.
